(19) United States
(12) Patent Application Publication
(10) Pub. No.: US 2021/0032584 A1
(43) Pub. Date: Feb. 4, 2021

(54) BLOOD VESSEL-MIMICKING MICROFLUIDIC CHIP FOR CELL CO-CULTURE AND USE THEREOF

(71) Applicant: SOGANG UNIVERSITY RESEARCH FOUNDATION, Seoul (KR)

(72) Inventors: Bong Geun CHUNG, Gyeonggi-do (KR); Seok Gyu MUN, Seoul (KR); Jong Min LEE, Seoul (KR); Hyung Woo CHOI, Chungcheongnam-do (KR); Jae Hyun LIM, Seoul (KR); Eun Joong KIM, Seoul (KR)

(21) Appl. No.: 16/666,577

(22) Filed: Oct. 29, 2019

(30) Foreign Application Priority Data
Jul. 30, 2019 (KR) 10-2019-0092581

(51) Int. Cl. C12M 3/06; B01L 3/00; C12N 5/071; G01N 33/50; G01N 33/543

(52) U.S. Cl. CPC C12M 23/16; B01L 3/502715; C12N 5/0691; B01L 2300/12; G01N 33/54346; B01L 2300/0861; G01N 33/5011

(57) ABSTRACT

The present disclosure provides a blood vessel-mimicking microfluidic chip for cell co-culture and a use thereof, wherein the microfluidic chip of the present disclosure is a microfluidic chip capable of co-culturing vascular endothelial cells and cancer cells, and can mimic normal vascular tissue, cancer tissue, and cancer-metastatic vascular tissue, and therefore can be widely used in studies associated with cancer, and especially, is suitable in studies on cancer metastasis, intravenous injection environments for cancer treatment, photothermal therapeutic effects on cancer cell, and the like.

### CROSS REFERENCE TO RELATED APPLICATION

This application claims priority to Korean Patent Application No. 10-2019-0092581 filed on Jul. 30, 2019. The entire disclosure of the application identified in this paragraph is incorporated herein by reference.

### FIELD

The present disclosure relates to a blood vessel-mimicking microfluidic chip for cell co-culture and a use thereof.

### BACKGROUND

Existing anticancer therapies mainly employ treatments, such as chemotherapy or radiotherapy, but the effects of the anticancer therapies are insufficient due to side effects thereof and the delivery of medicines is difficult since the medicines are inactivated or degraded before reaching target sites.

In-vitro studies for delivery of medicines in blood vessels have depended on large-sized cell culture in centimeter scales. The centimeter-scale is ineffective since such a scale can merely help the understanding of functions of the blood vessel system in the already diseased state, but not the understanding of functions of the pre-onset system, and cannot control drug delivery.

Meanwhile, cancer growth and proliferation can be studied through microfluidic chips capable of controlling microenvironments surrounding cells. The application of microfluidic chips to cancer cells can lead to an observation of various phenomena occurring in the human body, such as angiogenesis, immune responses, and cancer metastasis, and an observation of intercellular interactions, interactions between cells and cellular matrixes, and the like, thereby allowing systematic studies and in-vitro estimation of drugs and toxicity.

In recent years, microchips for isolating cancer cells from peripheral blood have been developed. Circulating tumor cells in the blood are causative cells of cancer metastasis. Although these cells were very difficult to isolate from cancer patients, the circulating tumor cells were effectively isolated using microchips. In addition to techniques of isolating cancer cells by using antigen-antibody interactions, techniques of continuously isolating circulating tumor cells from breast cancer patients by using hydrodynamic characteristics, such as size and density of cancer cells, have also been developed. These techniques allow the isolation of various kinds of circulating tumor cells, and thus can be applied to various cells. However, the metastasis of cancer cells and the treatment thereof were not considered in these microchips for detecting circulating tumor cells. Therefore, for precise mimicking and control of tumors and surrounding microenvironments thereof, there is required a three-dimensional co-culture of immune cells, vascular endothelial cells, and fibroblasts as well as cancer cells. This study requires an organic fusion of engineering research and cancer-related pathological knowledge.

### SUMMARY

The present inventors endeavored to develop a blood vessel-mimicking microfluidic chip for cell co-culture, the microfluidic chip being capable of mimicking normal blood vessels, cancer tissue, and a metastatic state of cancer cells into blood vessels and effectively analyzing photothermal therapeutic effects of nanoparticles. As a result, the present inventors verified that, by culturing cancer cells and vascular endothelial cells in a microfluidic chip having three cell culture channels and bridge channels connecting the cell culture channels, the cell culture channels mimic normal blood vessels, cancer tissue, and a metastatic state of cancer cells into blood vessels, respectively, and established that, by treating the microfluidic chip with nanoparticles, the photothermal therapeutic effects of the nanoparticles can be monitored, and thus the present inventors completed the present disclosure.

Accordingly, an aspect of the present disclosure is to provide a blood vessel-mimicking microfluidic chip for cell co-culture.

Another aspect of the present disclosure is to provide a method for analyzing a photothermal therapeutic effect on cancer cells by using the microfluidic chip of the present disclosure.

In accordance with an aspect of the present disclosure, there is provided a blood vessel-mimicking microfluidic chip for cell co-culture, the microfluidic chip including: (a) a first cell culture channel, a second cell culture channel, and a cell co-culture channel, as cell culture sections; and (b) bridge channels connected to the cell culture channels, wherein the cell co-culture channel is disposed between the first cell culture channel and the second cell culture channel and the first cell culture channel, the second culture channel, and the cell co-culture channel are connected through hollow tubular bridge channels.

The present inventors endeavored to develop a blood vessel-mimicking microfluidic chip for cell co-culture, the microfluidic chip being capable of mimicking normal blood vessels, cancer tissue, and a metastatic state of cancer cells into blood vessels and effectively analyzing photothermal therapeutic effects of nanoparticles. As a result, the present inventors verified that, by culturing cancer cells and vascular endothelial cells in a microfluidic chip having three cell culture channels and bridge channels connecting the cell culture channels, the cell culture channels mimic normal blood vessels, cancer tissue, and a metastatic state of cancer cells into blood vessels, respectively, and established that, by treating the microfluidic chip with nanoparticles, the photothermal therapeutic effects of the nanoparticles can be monitored.

The main characteristics of the present disclosure are that single-cell culture or cell co-culture can be attained through the three cell culture channels in the microfluidic chip and the delivery and control of nanocomposites having a photothermal therapeutic effect on cancer cells can be attained through the bridge channels in the microfluidic chip. Therefore, the microfluidic chip of the present disclosure mimics cancer cells, blood vessels, and a cancer-metastatic state into blood vessels through co-culture of cancer cells and vascular endothelial cells, and thus can mimic an intravenous injection environment for cancer therapy and can monitor a cancer-targeting photothermal therapeutic effect by using nanocomposites. Therefore, the co-culture of cancer cells and vascular endothelial cells in the microfluidic chip of the present disclosure can be widely applied in various studies associated with cancer.

In the blood vessel-mimicking microfluidic chip for cell co-culture of the present disclosure, the cell culture channels, as cell culture sections, may include a sample inlet and a sample outlet, and cells, cell culture solutions, samples necessary for analysis, nanoparticles showing a photothermal effect, and the like may be injected through the sample inlet.

According to an embodiment of the present disclosure, multiple (three or more) cell culture channels may be formed and may be arranged in multiple (three or more) columns or rows in the blood vessel-mimicking microfluidic chip for cell co-culture of the present disclosure. For instance, the first cell culture channel, the second cell culture channel, and the cell co-culture channel may be arranged in three columns, and the cell co-culture channel may be arranged between the first cell culture channel and the second cell culture channel.

The cell culture channels are connected through hollow tubular bridge channels in the blood vessel-mimicking microfluidic chip for cell co-culture of the present disclosure.

According to an embodiment of the present disclosure, the number of the bridge channels connected between the cell culture channels may be 1-20, 1-15, 1-10, 1-9, 1-8, 1-7, 3-20, 3-15, 3-10, 3-9, 3-8, 3-7, 5-20, 5-15, 5-10, 53-9, 5-8, or 5-7, and for example, 6.

Cells, cell culture solutions, samples necessary for analysis, nanoparticles showing a photothermal effect, and the like may be moved through the bridge channels in the microfluidic chip.

The microfluidic chip of the present disclosure may be manufactured of a polymer material selected from the group consisting of poly(dimethylsiloxane) (PDMS), polymethylmethacrylate (PMMA), polyacrylates, polycarbonates, polycyclic olefins, polyimides, and polyurethanes, and for example, the microfluidic chip may be manufactured of poly(dimethylsiloxane) (PDMS).

The microfluidic chip of the present disclosure may be bonded onto a plate facilitating optical measurement, which is selected from the group consisting of slide glass, crystal, and glass, and for example, the microfluidic chip may be bonded onto glass.

In the microfluidic chip of the present disclosure, different types of cells selected from the group consisting of cancer cells and vascular endothelial cells are cultured in the first cell culture channel and the second cell culture channel, respectively. For example, cancer cells are cultured in the first cell culture channel and vascular endothelial cells are cultured in the second cell culture channel, so that the first and second cell culture channels can mimic normal vascular tissue and cancer tissue in the same microfluidic chip, and cancer cells and vascular endothelial cells are cultured together in the cell co-culture channel between the first cell culture channel and the second cell culture channel, so that the cell co-culture channel can mimic cancer cell-metastatic vascular tissue. The bridge channels can mimic capillaries.

The cancer cells that can be cultured in the microfluidic chip of the present disclosure may be breast cancer cells, brain tumor cells, prostate cancer cells, rectal cancer cells, lung cancer cells, pancreatic cancer cells, ovarian cancer cells, bladder cancer cells, endometrial cancer cells, cervical cancer cells, liver cancer cells, kidney cancer cells, thyroid cancer cells, bone cancer cells, lymphoma cancer cells, or skin cancer cells, and for example, may be breast cancer cells, but are not limited thereto.

In accordance with another aspect of the present disclosure, there is provided a method for analyzing a photothermal therapeutic effect on cancer cells, the method including:

(a) preparing a blood vessel-mimicking microfluidic chip for cell co-culture, the microfluidic chip comprising: (i) a first cell culture channel, a second cell culture channel, and a cell co-culture channel, as cell culture sections; and (ii) bridge channels connected to the cell culture channels, wherein the cell co-culture channel is disposed between the first cell culture channel and the second cell culture channel and the first cell culture channel, the second culture channel, and the cell co-culture channel are connected through hollow tubular bridge channels;

(b) injecting vascular endothelial cells and cancer cells into the first cell culture channel and the second cell culture channel, respectively, and injecting vascular endothelial cells and cancer cells into the cell co-culture channel, followed by culture;

(c) injecting nanoparticles showing a photothermal effect into the first cell culture channel, the second cell culture channel, or the cell co-culture channel, followed by culture; and

(d) subjecting the microfluidic chip to laser irradiation to analyze the degrees of survival and death of the cancer cells.

As used herein, the term “photothermal therapy” (photothermal radiation or optical thermal therapy) is directed to a method for treatment of solid tumors, and typically includes a step of transforming absorbed light into local heat through a non-radioactive mechanism. The near-infrared (NIR) laser used in the photothermal therapy can deeply invade into the tissue with high spatial precision without damage to general biological tissue due to a low near-infrared absorption of general tissue.

The near-infrared laser is a laser beam having a wavelength region of 600-2000 nm, 600-1500 nm, 600-1300 nm, 600-1200 nm, 600-1100 nm, or 600-1000 nm.

According to an embodiment of the present disclosure, the photothermal therapeutic effect of nanoparticles is analyzed by culturing cancer cells and vascular endothelial cells in the blood vessel-mimicking microfluidic chip for cell co-culture of the present disclosure, injecting nanoparticles showing a photothermal effect into the cell culture channel, performing laser irradiation, and then checking the degrees of survival and death of cancer cells.

A cancer-targeting molecule may be conjugated to the nanoparticles. The conjugation of the cancer-targeting molecule can be expected for a superior photothermal therapeutic effect since the nanoparticle moves to bind to cancer cells. The cancer-targeting molecule is a molecule that specifically binds to a tumor-specific antigen or a tumor-associated antigen (TAA) expressed on the surface of cancer cells, and examples thereof include a compound, an antibody, or an antigen-binding fragment of the antibody. The antibody or antigen-binding fragment thereof may include scFv, Fab, or the like, but is not limited thereto.

According to an embodiment of the present disclosure, the nanoparticles used in the analysis of a photothermal therapeutic effect on cancer cells may be graphene oxide-based or carbon nanotube-based nanoparticles, and for example, may be reduced graphene oxide (rGO)-polyethylene glycol (PEG)-folic acid (FA) in which folic acid as a cancer-targeting molecule is conjugated.

According to another embodiment of the present disclosure, the nanoparticles used in the analysis of a photothermal therapeutic effect on cancer cells may be gold nanorods, gold nanocapsules, magnetic particles (i.e., iron oxide nanoparticles), quantum dots, or nanoparticles of gold-coated polymers (e.g., hyaluronic acid, cellulose, dextran).

The method for analyzing a photothermal therapeutic effect on cancer cells of the present disclosure is directed to the analysis of a photothermal therapeutic effect on cancer cells by using the foregoing blood vessel-mimicking microfluidic chip for cell co-culture, and thus the overlapping contents therebetween are omitted to avoid excessive complexity of the specification due to repetitive descriptions thereof.

According to the present disclosure, there are provided a blood vessel-mimicking microfluidic chip for cell co-culture and a use thereof. The microfluidic chip of the present disclosure is a microfluidic chip capable of co-culturing vascular endothelial cells and cancer cells, and can mimic normal vascular tissue, cancer tissue, and cancer-metastatic vascular tissue, and therefore can be widely used in studies associated with cancer, and especially, is suitable in studies on cancer metastasis, intravenous injection environments for cancer treatment, photothermal therapeutic effects on cancer cell, and the like.

### BRIEF DESCRIPTION OF THE DRAWINGS

The above and other aspects, features and advantages of the present disclosure will be more apparent from the following detailed description taken in conjunction with the accompanying drawings, in which:

FIG. 1a is a schematic view showing a blood vessel-mimicking microfluidic chip for cell co-culture, the microfluidic chip being composed of three cell culture channels and bridge channels;

FIG. 1b is a schematic view of a microfluidic chip composed of three cell culture channels for a photothermal effect by NIR irradiation and bridge channels;

FIG. 1c is a microscopic image of a microfluidic chip filed with a fluorescent dye;

FIG. 1d is an actual image of a microfluidic chip;

FIGS. 2a, 2b, 2c, 2d and 2e are schematic diagrams of a synthesis procedure of rGO-PEG-FA;

FIG. 3a is a TEM image of rGO-PEG-FA;

FIG. 3b shows FT-IR analysis results of GO-COOH, rGO-PEG, and rGO-PEG-FA;

FIG. 3c shows UV-Vis analysis results of rGO-PEG and rGO-PEG-FA;

FIG. 3d shows ZETA analysis results of GO-COOH, rGO-PEG, and rGO-PEG-FA;

FIG. 3e shows the results of analyzing temperatures of culture media containing various concentrations of rGO-PEG-FA in the presence of NIR laser irradiation;

FIG. 4a shows confocal microscopic images of HUVEC cells and MDA-MB-231 cells treated with rGO-PEG (30 μg/ml);

FIG. 4b shows confocal microscopic images of the cells treated with rGO-PEG-FA (folic acid conjugation) for breast cancer targeting;

FIG. 5 shows confocal microscopic images after the microfluidic chip for cell co-culture was treated with rGO-PEG-FA (30 μg/ml) ((d)-(f) are high-resolution images in the HUVEC channel, co-culture channel, and MDA-MB-231 channel (scale bar: 50 μm));

FIG. 6a shows the results of evaluating toxicity of rGO-PEG-FA in HUVEC cells;

FIG. 6b shows the results of evaluating toxicity of rGO-PEG-FA in MDA-MB-231 cells;

FIG. 7a shows confocal microscopic images of HUVEC cells (green) and MDA-MB-231 cells (red) in cell culture dishes (scale bar: 100 μm);

FIG. 7b is a confocal microscopic image of HUVEC cells (green) and MDA-MB-231 cells (red) in a microfluidic chip. Scale bar: 100 μm;

FIG. 8a shows Live (green)/Dead (red) fluorescent images before and after NIR irradiation after HUVEC cells and MDA-MB-231 cells were treated with rGO-PEG-FA (30 μg/ml) (a/d, b/e, and c/f represent 20-fold magnification images in the HUVEC channel, co-culture channel, and MDA-MB-231 channel); and

FIG. 8b shows the results of analyzing cell viability by photothermal therapy in a microfluidic chip.

### DETAILED DESCRIPTION

Hereinafter, the present disclosure will be described in more detail with reference to examples. These examples are only for illustrating the present disclosure more specifically, and it will be apparent to those skilled in the art that the scope of the present disclosure is not limited by these examples.

### EXAMPLES

### Example 1: Fabrication of Blood Vessel-Mimicking Microfluidic Chip

1-1. Fabrication of Blood Vessel-Mimicking Microfluidic Chip

For fabrication of a blood vessel-mimicking microfluidic chip capable of evaluating a photothermal therapeutic effect of a functional nanocomposite (rGO-PEG-FA), chambers and bridge channels were manufactured through two-step photolithography by using a known method. For fabrication of the blood vessel-mimicking microfluidic chip, the chambers and bridge channels were designed using AutoCAD program. To manufacture the bridge channels, SU-8 25 photoresist was spin-coated on a silicon wafer (1000 rpm, 60 s, and 40 Gm in thickness). To manufacture chambers, SU-8 100 was spin-coated on the SU-8 25 photoresist-patterned substrate (3,000 rpm, 60 s, and 250 Gm in thickness). The poly(dimethylsiloxane) (PDMS) precursor solution was poured on the photoresist-patterned silicon wafer, and PDMS-based 3D microfluidic co-culture device was bonded onto glass slides using oxygen plasma treatment (Femto Science, Korea).

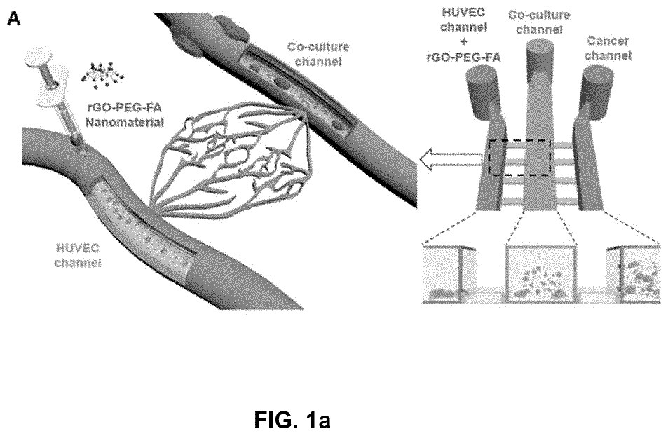

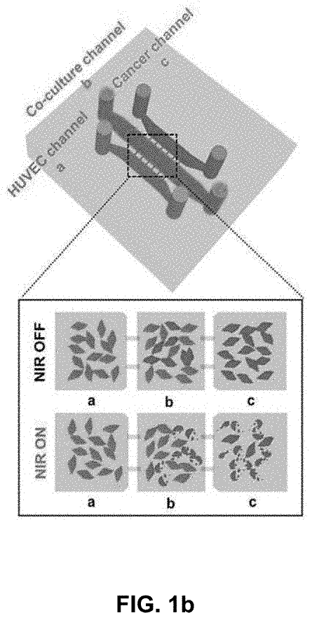

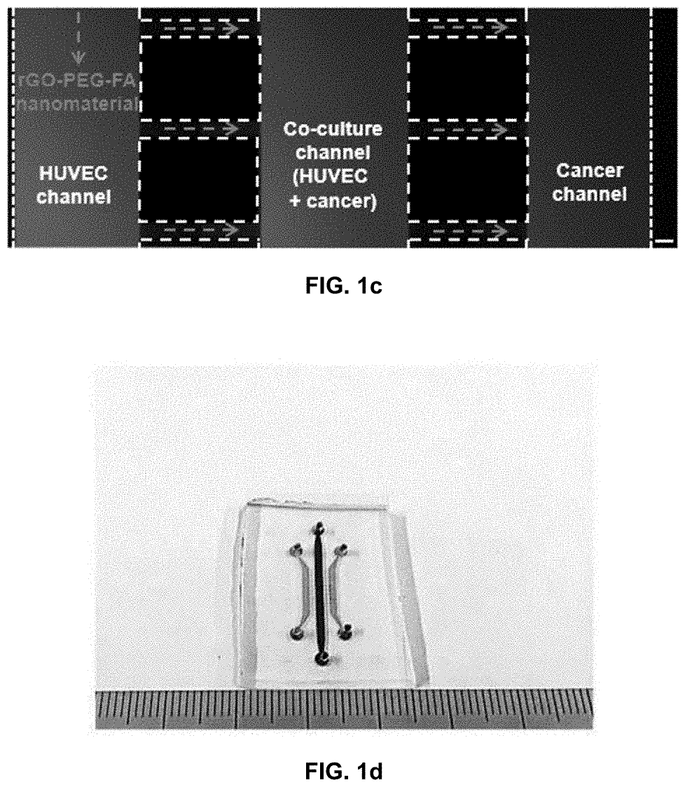

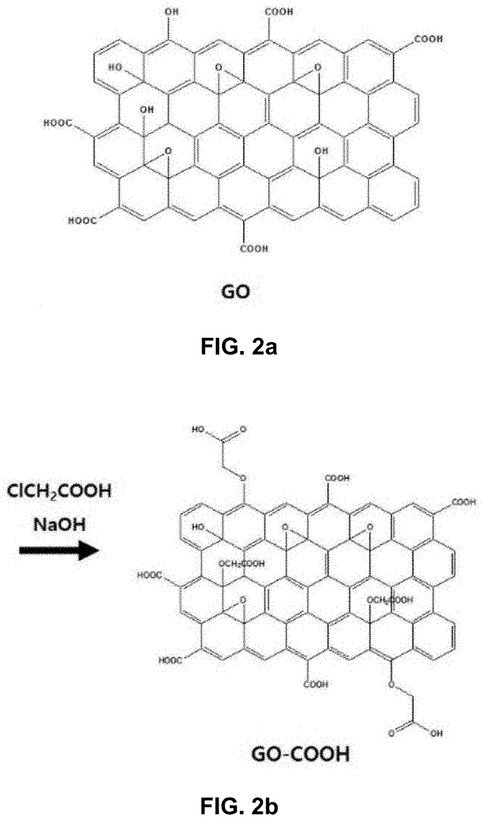

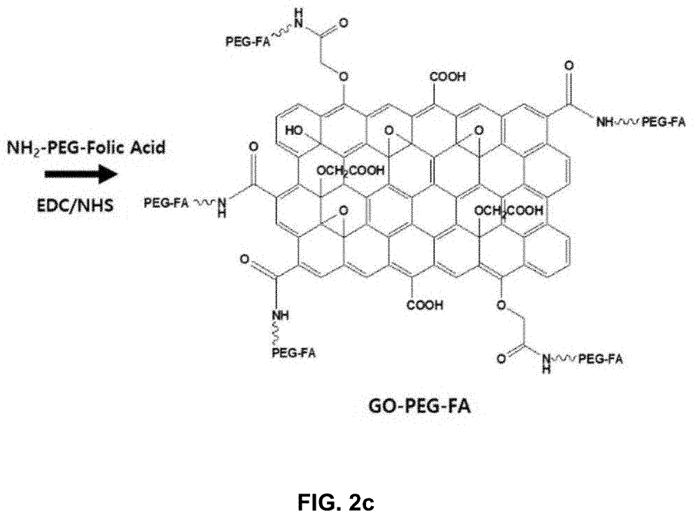

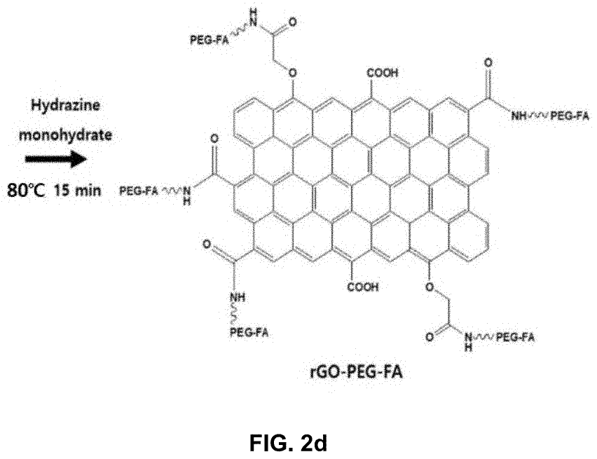

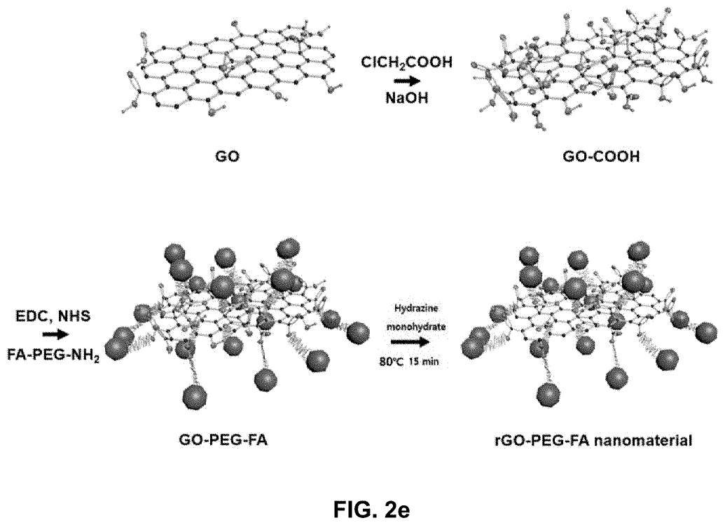

The microfluidic chip is composed of three cell culture channels arranged in three columns and six bridge channels connecting each cell culture channels (FIGS. 1a and 1b).

1-2. Characteristics of Blood Vessel-Mimicking Microfluidic Chip

The left cell culture channel mimics a normal blood vessel by culturing human umbilical vein endothelial cells (HUVEC) therein, the right cell culture channel mimics cancer tissue by culturing breast cancer cells (MDA-MB-231) therein, and the middle cell culture channel mimics a metastatic state of cancer cells into a blood vessel by co-culturing human umbilical vein endothelial cells (HUVEC) and breast cancer cells (MDA-MB-231) therein. In addition, the bridge channels connecting the respective cell culture channels are channels through which a functional nanocomposite can move, and the bridge channels mimicked capillaries existing between blood vessels and tissue. For observation of the movement of the functional nanocomposite through the bridge channels, a fluorescent dye (fluorescein isothiocyanate-dextran) was used. It was observed that the nanocomposite can be diffused in the microfluidic chip.

The cells were injected into the microfluidic chip fabricated in the present disclosure by using a pipette tip. The prior method (Korean Patent No. 10-1709312) discloses that cells were injected into different microchannels by using physical walls of a hydrogel, but in the present disclosure, only a pipette tip was used in the inlet and outlet to form a pressure difference without the introduction of an external substance, such as a hydrogel, so that desired cells could be seeded in desired locations in the proposed channels.

According to a simple description of the method, the attachment of different types of cells was allowed in the chip while the dates of injection were set to be different from each other. For instance, on the first day, in the process of seeding human umbilical vein endothelial cells into the HUVEC channel (on the leftmost), a pipette tip was inserted into the inlet and outlet of the cancer channel to create a pressure difference, and thus HUVECs did not move to the cancer channel but moved to only the co-culture channel in the procedure of seeding the cells. After one day, the breast cancer cells were injected into the cancer channel. The injection of the breast cancer cells was also carried out in the same manner, that is, a pipette tip was inserted into the inlet and outlet of the HUVEC channel, so that the breast cancer cells were injected and attached to only the middle co-culture channel. The injection of cells at an interval of one day provided a condition in which the respective injected cells could be attached in the respective proposed channels. Such cell injection and fixation using pressure differences through a pipette tip or the like has an advantage that a desired combination of cells (separate culturing or co-culturing two different types of cells) can be made in the proposed channels without the introduction of an external substance.

As described above, the microfluidic chip of the present disclosure can make a desired combination of cells in the proposed channels through cell injection and attachment using the pressure differences formed by the pipette chips, and using this, the HUVEC channel, the co-culture channel, and the MDA-MB-231 channel can be made from the leftmost channel. Here, the HUVEC channel mimicked normal tissue (vein), the co-culture channel mimicked cancer-metastatic tissue, the MDA-MB-231 channel mimicked cancer tissue, and the bridge channels mimicked capillaries. Such mimicking can lead to a precise mimic of the cancer environment in the in-vitro environment. The introduction of the functional nanocomposite was implemented through the HUVEC channel on the leftmost side, which mimics intravenous injection. The functional nanocomposite was introduced through the HUVEC channel, and gradually moved to the co-culture channel mimicking cancer-metastatic tissue and to the MDA-MB-231 channel mimicking primary carcinoma, through the bridge channels mimicking capillaries. This can precisely mimic that upon intravenous injection, a drug was introduced through a vein and delivered into the cancer tissue in which cancer metastasis occurred.

### Example 2: Synthesis of Functional Nanocomposite (rGO-PEG-FA)

Graphene oxide (GO) was exposed to ClCH₂COOH and NaOH to modify the surface OH group into the COOH (carboxyl) group, and the polyethylene glycol (PEG) for improving dispersion stability in an aqueous solution and folic acid (FA) capable of targeting particular cancer cells were stirred for 18 hours by using EDC, NHS reaction, thereby conjugating PEG and FA to the carboxyl group of graphene. After the reaction, unreacted materials were removed using the 6-8 kDa dialysis membrane, and the resultant product was placed in the 0.05 v/v % hydrazine solution, and reduced at 80° C. (reduced graphene oxide, rGO).

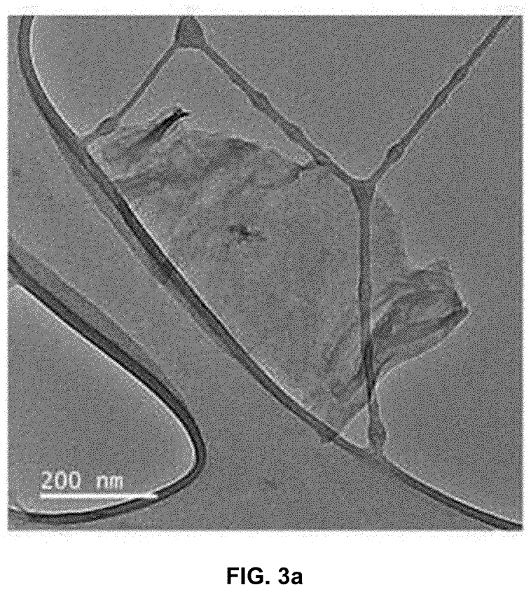

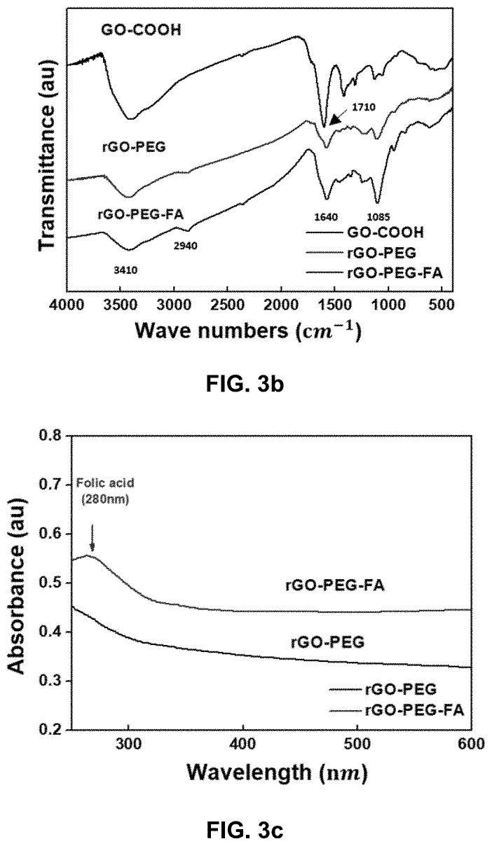

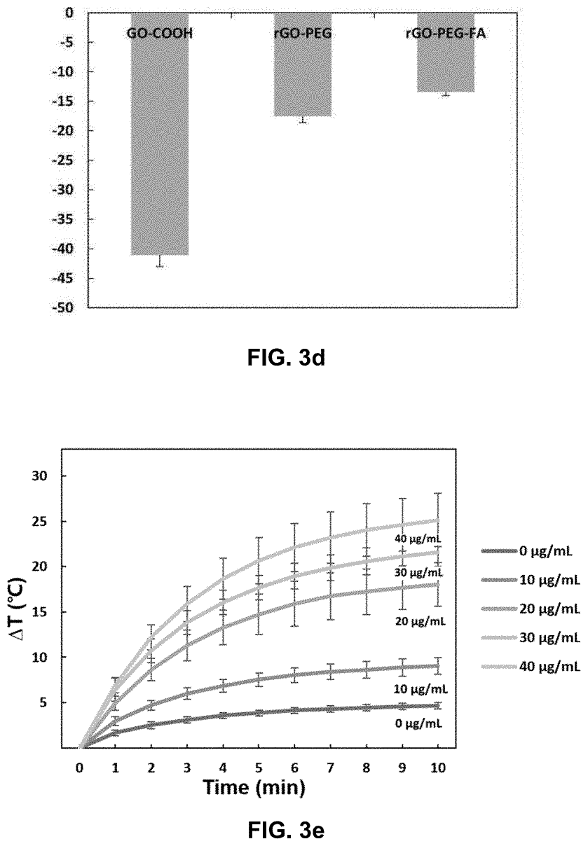

For the evaluation of spectroscopic characteristics of the synthesized reduced graphene oxide (rGO)-polyethylene glycol (PEG)-folic acid (FA), Fourier-transform infrared spectroscopy (FT-IR) and UV-visible spectroscopy were used. As a result, a broad peak was shown at 3410 cm⁻¹ due to OH in the FA structure, and an absorption wavelength was shown at 1085 cm⁻¹ due to the C—O—C structure of PEG. COOH of the GO surface and NH₂ form CONH bonds due to EDC and NHS, and thus the peak of COOH at 1710 cm⁻¹ was not observed, and a peak was observed at 1640 cm⁻¹ (FIG. 3b). Since FA has an absorption wavelength of around 320 nm, rGO-PEG-FA showed a shifted absorption wavelength of about 280 nm when compared with rGO-PEG, indicating the successful modification of FA (FIG. 3c). As a result of measuring the zeta potential of the synthesized rGO, GO-COOH showed a strong negative charge of −40 mV due to surface COOH, showed a decrease of about −18 mV due to a covalent linkage with mPEG-NH₂, and had a charge of −17 mV due to the FA-PEG-NH₂ modification (FIG. 3d).

The synthesized rGO-PEG-FA with various concentrations were subjected to an 808 nm NIR laser at a power density of 2 W/cm² to measure a photothermal effect by using a thermocoupler. The results verified that as the concentration increased, a stronger photothermal effect was shown (FIG. 3e).

### Example 3: Analysis of Cellular Uptake of Functional Nanocomposite (rGO-PEG-FA)

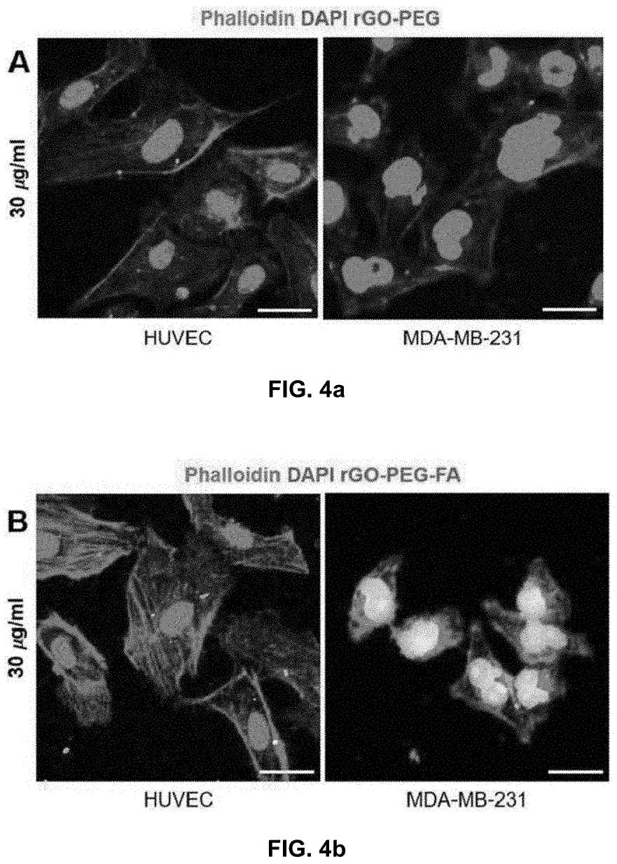

For confirmation of targeting and cellular uptake of the functional nanocomposite (rGO-PEG-FA) on cancer cells (MDA-MB-231), human umbilical vein endothelial cells and breast cancer cells were seeded at 2×10⁴ per well on plates for a confocal laser microscope, and the cells were cultured for 3 days while the culture medium was exchanged once a day, and then the cells were treated with functional nanocomposites (rGO-PEG and rGO-PEG-FA) at a concentration of 30 μg/ml each for 4 hours. The functional nanocomposites were visualized by conjugating FITC (green) to rGO-PEG and rGO-PEG-FA. The cells treated with the functional nanocomposites were stained with Phalloidin 594 (red) overnight and DAPI (blue) for 1 hour. Thereafter, images were taken by a confocal laser microscope. Since the cellular uptake of rGO-PEG did not occur, the uptake thereof was not also observed in the human umbilical vein endothelial cells and breast cancer cells (FIG. 4a). The cellular uptake of rGO-PEG-FA occurred by folate receptors in the breast cancer cells and thus the expression of FITC fluorescence was observed in the cytosol, whereas the human umbilical vein endothelial cells has no folate receptors and thus the expression of FITC fluorescence through the cellular uptake was not observed in the human umbilical vein endothelial cells (FIG. 4b).

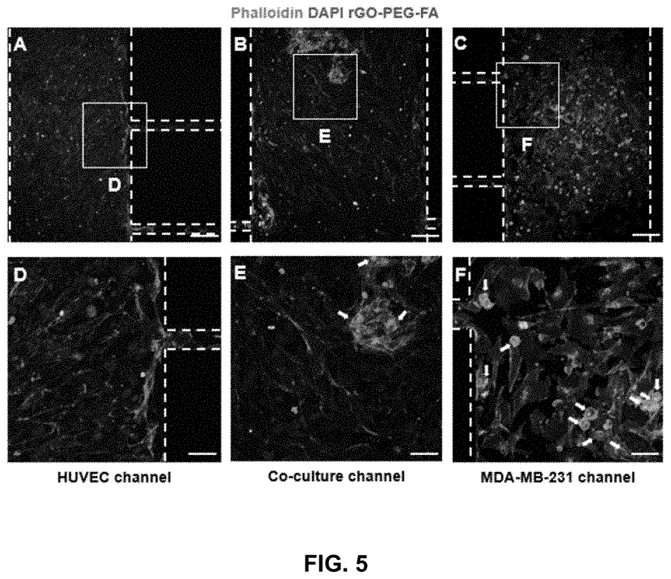

As described above, the cellular uptake of the functional nanocomposite was also observed in each channel on the microfluidic chip as well as cell culture plates. The functional nanocomposite could be observed through a microscope by conjugation to the FITC fluorescence dye (green), and the functional nanocomposite was injected through the HUVEC channel to mimic the intravenous injection. In FIG. 5, (d) to (f) show high-resolution images of the respective channels. The cellular uptake results were shown in a gradient form similar to the results in the cell culture plates. In the HUVEC channel, the cellular uptake of the nanocomposite did not occur in spite of the introduction of the nanocomposite since only human umbilical vein endothelial cells existed, and therefore, the expression of FITC fluorescence (green) was not observed. On the contrary, in the MDA-MB-231 channel in which the nanocomposite was injected through diffusion, the cellular uptake was high since only breast cancer cells existed, and therefore, the FITC fluorescence (green) was observed in the cells existing in the channel. In the co-culture channel, the breast cancer cells expressing FITC fluorescence (green) partially existed, and therefore, the expression of fluorescence was partially observed.

### Example 4: Evaluation of Toxicity of Functional Nanocomposite (rGO-PEG-FA)

The toxicity of the functional nanocomposite (rGO-PEG-FA) was evaluated in human umbilical vein endothelial cells and breast cancer cells before and after NIR laser irradiation. Respective cells were seeded at a density of 1×10⁴ per well in 96-well plates, and treated with the functional nanocomposite at concentrations of 0 μg/ml, 10 μg/ml, 20 μg/ml, 30 μg/ml, and 40 μg/ml for 4 hours. As for the evaluation of toxicity before NIR laser irradiation, the cells were treated with the functional nanocomposite for 4 hours, and then immediately the cell viability was calculated by the CCK-8 Kit. As for the evaluation of toxicity after NIR laser irradiation, the cells were treated with the functional nanocomposite for 4 hours, and thereafter irradiated with an 808 nm NIR laser at an intensity of 2 W/cm² for 10 minutes, and then the cell viability was calculated by the CCK-8 Kit.

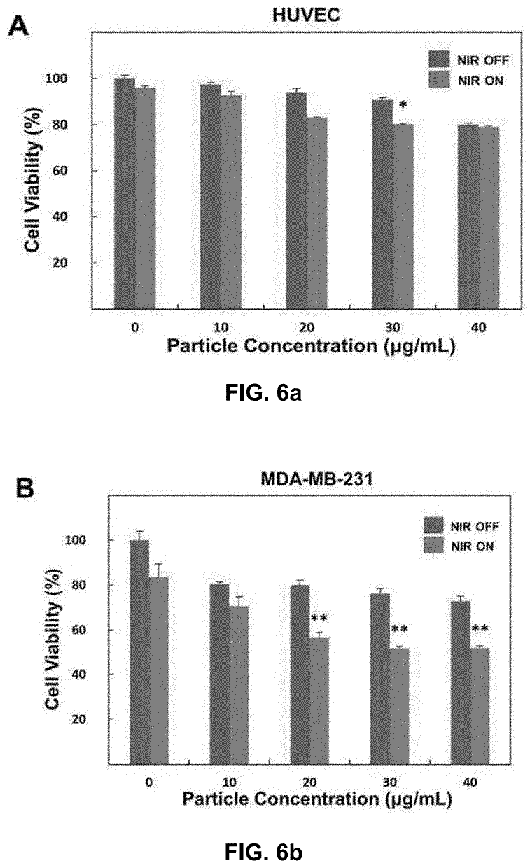

In the human umbilical vein endothelial cells, before NIR laser irradiation, the cell viability was 91% at 30 μg/ml but was significantly decreased to 80% at 40 μg/ml, and thus the functional nanocomposite was determined to be toxic at 40 μg/ml or higher; and after NIR laser irradiation, the cell viability was 80% at 30 μg/ml, indicating a decrease of only 10% compared with the cell viability before NIR laser irradiation (FIG. 6a). In the breast cancer cells, before NIR laser irradiation, as the concentration increased, the cell viability decreased to 76% at 30 μg/ml, and decreased to 73% at 40 μg/ml. The cell viability at 30 μg/ml decreased to 52% after NIR laser irradiation from 76% before NIR laser irradiation (FIG. 6b). Considering that the cell viability of the human umbilical vein endothelial cells decreased to 80% at 40 μg/ml before the laser irradiation, the functional nanocomposite was determined to be toxic at 40 μg/ml, and therefore, the concentration of the functional nanocomposite was optimized to 30 μg/ml.

### Example 5: Investigation of Photothermal Therapeutic Effect of Functional Nanocomposite (rGO-PEG-FA)

5-1. Evaluation of Cell Viability in Microfluidic Chip

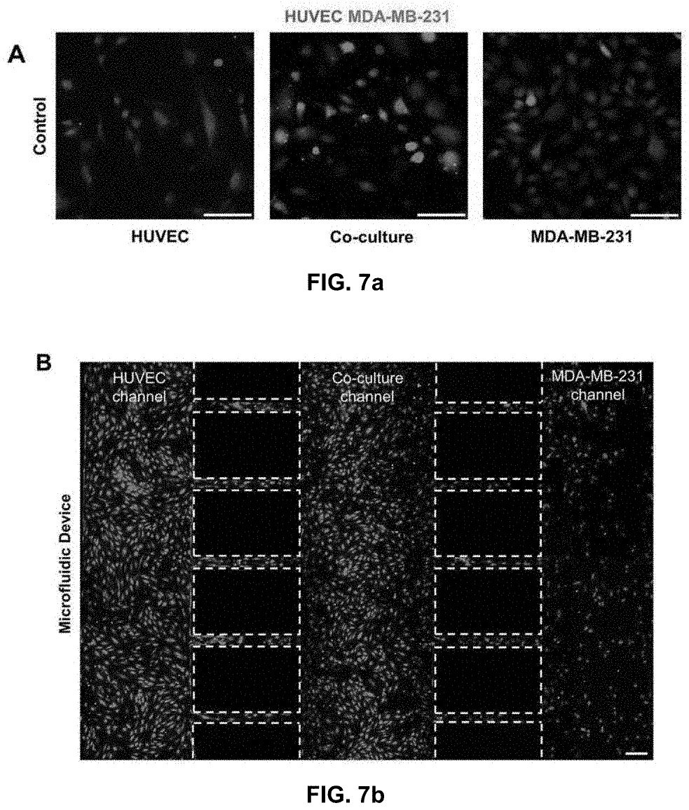

To investigate where the cells were injected, fixed, and cultured, the human umbilical vein endothelial cells were stained with CFSE (green), and then 2×10⁵ cells were seeded through the inlet of the left channel and 1×10⁵ cells were seeded in the middle co-culture channel, and then cultured for one day. After the human umbilical vein endothelial cells were cultured for one day, the breast cancer cells were stained with Far Red, and then 2×10⁵ cells were seeded through the inlet of the right channel and 1×10⁵ cells were seeded in the co-culture channel. The culture medium was exchanged once a day for 3 days, and then for confirmation of the cultured cells, observation was conducted by a confocal microscope. The results confirmed that the cells were viable for 3 days and existed in the respective proposed channels (FIG. 7b). For the investigation of cell viability, the same amounts of the cells injected into the microfluidic chip were cultured in cell culture dishes for 3 days while the culture medium was exchanged once a day. Thereafter, it was confirmed that the cells were viable through comparison among the confocal microscopic images in the microfluidic chip (FIG. 7a).

5-2. Evaluation of Photothermal Therapeutic Effect of Functional Nanocomposite

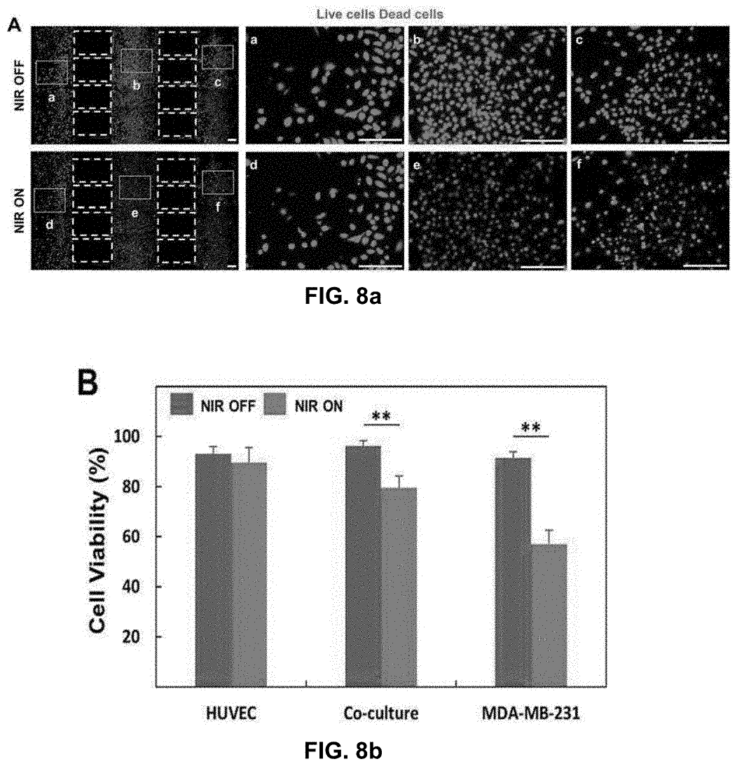

To investigate a photothermal therapeutic effect of the manufactured nanocomposite, the cells were incubated in the microfluidic chip for 3 days in the same manner as above except that cell staining was omitted. After cell culture, the nanocomposite was treated at a concentration of 30 μg/ml for 4 hours in the human umbilical vein endothelial cell channel. After treatment with the nanocomposite, the cells were stained with the live/dead assay, and then images before 808 NIR laser irradiation were taken using a fluorescent microscope. Thereafter, the cells were irradiated with NIR laser at an intensity of 2 W/cm² for 10 minutes, and the images of the chip irradiated with laser were taken by using a fluorescent microscope (FIG. 8a). The images before and after NIR laser irradiation were analyzed for a fluorescent intensity by using the Image J program, and the cell viability was calculated through fluorescent intensity.

The cell viability in the human umbilical vein endothelial cells was 93% and 90% before and after NIR irradiation, respectively, which were almost not different from each other. The cell viability in the co-culture channel was 96% and 79% before and after NIR irradiation, which were different due to the presence of breast cancer cells. The cell viability in the breast cancer cell channel was 92% and 57% before and after NIR irradiation, respectively, which were greatly different from each other (FIG. 8b). Overall, the cell viability before NIR irradiation was 93%, 96%, and 92% in the umbilical vein endothelial cell channel, the co-culture channel, and the breast cancer cell channel, respectively, which were not different among each other, and the cell viability after NIR irradiation was 90%, 79%, and 57% in the umbilical vein endothelial cell channel, the co-culture channel, and the breast cancer cell channel, respectively, which were greatly different among each other. It can be construed that the difference in cell viability was made by selective uptake of the functional nanocomposite into only the breast cancer cells through targeting of breast cancer cells by folic acid conjugated to the functional nanocomposite and the photothermal therapeutic effect by NIR laser irradiation.

What is claimed is:

1. A blood vessel-mimicking microfluidic chip for cell co-culture, the microfluidic chip comprising:
(a) a first cell culture channel, a second cell culture channel, and a cell co-culture channel, as cell culture sections; and
(b) bridge channels connected to the cell culture channels,
wherein the cell co-culture channel is disposed between the first cell culture channel and the second cell culture channel and the first cell culture channel, the second culture channel, and the cell co-culture channel are connected through hollow tubular bridge channels.

2. The microfluidic chip of claim 1, wherein different types of cells selected from the group consisting of cancer cells and vascular endothelial cells are cultured in the first cell culture channel and the second cell culture channel, respectively.

3. The microfluidic chip of claim 1, wherein cancer cells and vascular endothelial cells are co-cultured in the cell co-culture channel.

4. The microfluidic chip of claim 1, wherein the microfluidic chip is manufactured of a polymer material selected from the group consisting of poly(dimethylsiloxane) (PDMS), polymethylmethacrylate (PMMA), polyacrylates, polycarbonates, polycyclic olefins, polyimides, and polyurethanes.

5. The microfluidic chip of claim 1, wherein the microfluidic chip is bonded onto a plate facilitating optical measurement, which is selected from the group consisting of slide glass, crystal, and glass.

6. A method for analyzing a photothermal therapeutic effect on cancer cells, the method comprising:
(a) preparing a blood vessel-mimicking microfluidic chip for cell co-culture, the microfluidic chip comprising: (i) a first cell culture channel, a second cell culture channel, and a cell co-culture channel, as cell culture sections; and (ii) bridge channels connected to the cell culture channels, wherein the cell co-culture channel is disposed between the first cell culture channel and the second cell culture channel and the first cell culture channel, the second culture channel, and the cell co-culture channel are connected through hollow tubular bridge channels;
(b) injecting vascular endothelial cells and cancer cells into the first cell culture channel and the second cell culture channel, respectively, and injecting vascular endothelial cells and cancer cells into the cell co-culture channel, followed by culture;
(c) injecting nanoparticles showing a photothermal effect into the first cell culture channel, the second cell culture channel, or the cell co-culture channel, followed by culture; and
(d) subjecting the microfluidic chip to laser irradiation to analyze the degrees of survival and death of the cancer cells.

7. The method of claim 6, wherein the nanoparticles are graphene oxide-based nanoparticles or gold nanoparticle-based nanoparticles.

8. The method of claim 7, wherein a cancer-targeting molecule is conjugated to the nanoparticles.

9. The method of claim 7, wherein the graphene oxide-based nanoparticles are formed of reduced graphene oxide (rGO)-polyethylene glycol (PEG)-folic acid (FA).
